(19) United States
(12) Patent Application Publication
(10) Pub. No.: US 2026/0242318 A1
(43) Pub. Date: Aug. 20, 2026

(54) METHOD FOR PREPARING HONOKIOL DERIVATIVE AND INTERMEDIATE THEREOF

(71) Applicant: BEIJING JINRUI FOUNDATION PHARMACEUTICAL TECHNOLOGY CO., LTD., Beijing (CN)

(72) Inventors: Jianhua Ge, Beijing (CN); Fengchun Chen, Beijing (CN); Wenwen Wang, Beijing (CN); Rui Wang, Beijing (CN); Yingying Zhong, Beijing (CN)

(21) Appl. No.: 19/646,614

(22) Filed: Apr. 14, 2026

(30) Foreign Application Priority Data
Apr. 15, 2025 (CN) 202510466461.6

(51) Int. Cl. C07C 41/18; C07C 37/055; C07C 45/64

(52) U.S. Cl. CPC C07C 41/18; C07C 37/055; C07C 45/64

(57) ABSTRACT

A method for preparing a honokiol derivative and an intermediate thereof is provided. The method includes: subjecting a compound M2 to a Wittig reaction with ethyltriphenylphosphonium bromide under basic conditions to prepare a compound of Formula I, subjecting the compound of Formula I to an allylation reaction to prepare a compound M3, subjecting the compound M3 to MOM deprotection under acidic conditions to prepare a compound M4, and subjecting the compound M4 to a rearrangement reaction to prepare a compound of Formula II. The starting materials of the method are inexpensive and readily available, with low cost, simple steps, mild reaction conditions, low purification requirements, high purity, high yield, environmental friendliness, safety, and suitability for industrial production, having significant application value.

### CROSS-REFERENCE TO RELATED APPLICATION

The present application claims priority from Chinese Patent Application No. 202510466461.6 filed on Apr. 15, 2025, the contents of which are incorporated herein by reference in their entirety.

### FIELD OF THE INVENTION

The present disclosure relates to the field of medicinal chemistry, and particularly to a method for preparing a honokiol derivative and an intermediate thereof.

### BACKGROUND OF THE INVENTION

The ability to induce angiogenesis is a prerequisite for the generation, infiltration, and metastasis of malignant tumors. Tumor cells themselves, as well as inflammatory cells (mainly macrophages) infiltrating the tumor tissue and its surroundings, can produce a class of angiogenic factors, such as vascular endothelial growth factor (VEGF) and basic fibroblast growth factor (b-FGF). These angiogenic factors promote the division of vascular endothelial cells and the sprouting growth of capillaries. The newly formed capillaries not only provide nutrients for tumor growth but also create favorable conditions for tumor metastasis. Therefore, angiogenesis inhibitors can be used for the prevention and treatment of most tumors, such as lung cancer, liver cancer, colorectal cancer, and hemangioma.

Honokiol, 3′,5-di-2-propenyl-1,1′-biphenyl-2,4′-diol (C₁₈H₁₈O₂), exhibits good vascular inhibition and anti-tumor effects, and also possesses anti-inflammatory, antibacterial, anti-pathogenic microorganism, anti-ulcer, antioxidant, anti-aging, and cholesterol-lowering activities.

It has been reported that honokiol derivatives have similar vascular inhibition and anti-tumor effects as honokiol. Chinese Patent Application Publication No. CN106278829A discloses honokiol derivatives of the following structure, wherein R₁ and R₂ are independently allyl, (E)-propenyl, (Z)-propenyl, formyl, 2,3-dihydroxypropyl, 3-hydroxy-2-methoxypropyl, 2-hydroxy-3-methoxypropyl, or 2,3-epoxypropyl; and R₁ and R₂ are not both allyl.

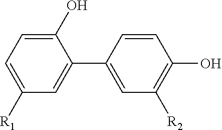

These honokiol derivatives are used for anti-tumor and angiogenesis inhibition, exhibiting effects comparable to honokiol, and have broad application prospects in drugs for treating cancer and angiogenesis-related diseases.

Chinese Patent Publication No. CN109846863B discloses the use of honokiol for preventing or treating Hashimoto's thyroiditis and/or its complications in humans, wherein honokiol can also be replaced by honokiol structural analogs having the same active groups.

Chinese Patent Application Publication No. CN106278829A employs a degradation process starting from honokiol to prepare the honokiol derivatives, which has a long degradation period. The work-up procedure involves high-speed counter-current chromatography followed by column chromatography separation and finally purification by semi-preparative high-performance liquid chromatography, resulting in complex procedures and low yields.

Bernd Schmidt et al., in the paper titled “Synthesis of Magnaldehydes B and E and Dictyobiphenyl B by Microwave-Promoted Cross-Coupling of Boronophenols” disclosed a synthetic method for honokiol derivatives as shown in the scheme below. The reaction involves six steps: bromination, coupling, protection, allylation, rearrangement, and deprotection. The palladium-carbon (Pd/C) coupling requires a high temperature of 150° C., and the allyl rearrangement temperature reaches as high as 250° C., resulting in harsh reaction conditions. The use of microwave irradiation is not conducive to scale-up production. The MOM deprotection

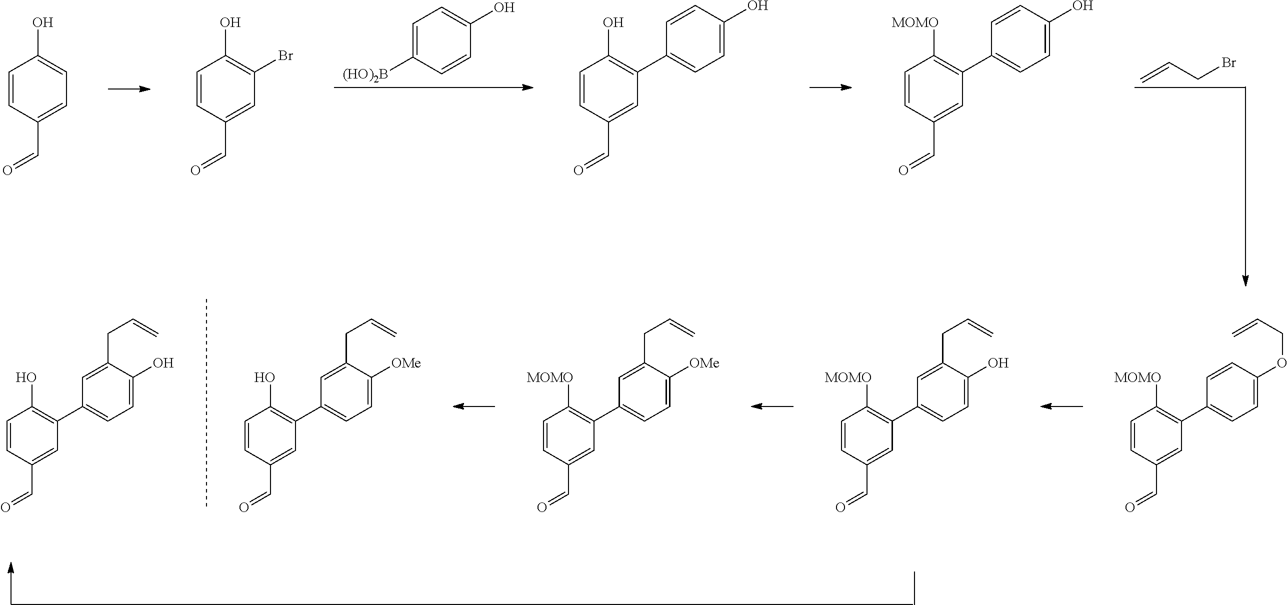

uses CF₃CO₂H, which leads to reactions that are difficult to control, produce complex mixtures, and are environmentally unfriendly, unsafe, and unsuitable for scale-up. Furthermore, performing the coupling reaction before protecting the phenolic hydroxyl groups is easy to generate by-products, therefore, the yield is reduced.

### SUMMARY OF THE INVENTION

An object of the present disclosure is to provide a method for preparing a honokiol derivative and an intermediate thereof, which features simple steps, low cost, mild reaction conditions, environmental friendliness, safety and suitability for industrial production.

The present disclosure provides a method for preparing a compound of Formula I as below, comprising subjecting a compound M2 as below to a Wittig reaction with ethyltriphenylphosphonium bromide under basic conditions to prepare the compound of Formula I;

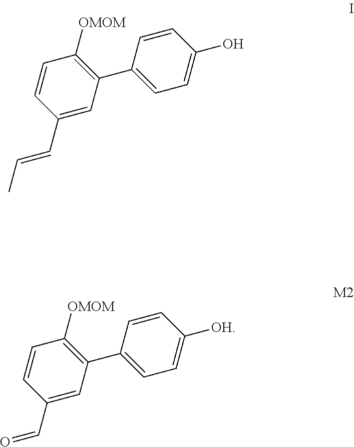

Preferably, the compound M2 is prepared by the following steps:
(1) reacting a compound SM1 with bromomethyl methyl ether to prepare a compound M1;

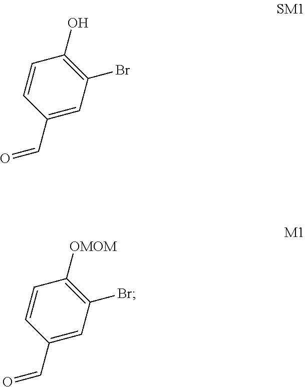

(2) reacting the compound M1 with a compound SM2 in a coupling reaction to prepare the compound M2;

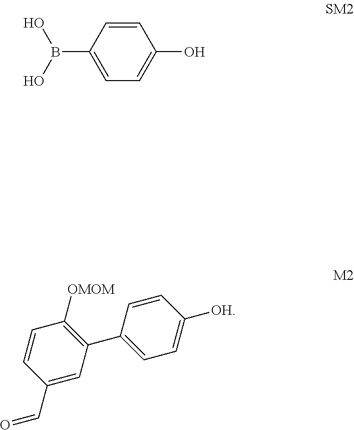

Preferably, in a reaction of step (1), triethylamine is used as a base.

Preferably, the molar ratio of the compound SM1, the bromomethyl methyl ether, and the base is 1:(1.0 to 1.4):(1.5 to 2.0), more preferably 1:1.1:1.5.

Preferably, in a reaction of step (1), tetrahydrofuran is used as a solvent.

Preferably, in a reaction of step (1), the reaction temperature is −5° C. to 5° C., and the reaction time is 3 to 6 h.

Preferably, step (1) employs a purification method of trituration, more preferably trituration with a mixed solvent of petroleum ether and ethyl acetate.

Preferably, in the coupling reaction of step (2), Pd(OAc)₂ is used as a catalyst, 1,1′-bis(diphenylphosphino)ferrocene is used as a ligand.

Preferably, in the coupling reaction of step (2), the base comprises at least one selected from the group consisting of potassium carbonate, cesium carbonate and potassium acetate, more preferably potassium carbonate.

Preferably, the molar ratio of the compound M1, the base, the catalyst, and the ligand is 1:(2 to 3):(0.09 to 0.12):(0.09 to 0.12).

Preferably, in step (2), the molar ratio of the compound M1 to the compound SM2 is 1:(1.0 to 1.5), more preferably 1:1.4.

Preferably, in step (2), a solvent for the coupling reaction comprises a mixed solvent of N,N-dimethylformamide and water, a mixed solvent of 1,4-dioxane and water, or a mixed solvent of tetrahydrofuran and water, more preferably is a mixed solvent of tetrahydrofuran and water.

Preferably, in step (2), the coupling reaction temperature is 50 to 70° C., more preferably 60 to 65° C., and the coupling reaction time is 15 to 25 h, more preferably 20 h.

Preferably, step (2) employs a purification method of trituration, more preferably trituration with a mixed solvent of petroleum ether and ethyl acetate.

Preferably, the base comprises at least one selected from the group consisting of potassium tert-butoxide, sodium tert-butoxide, and lithium diisopropylamide, more preferably potassium tert-butoxide.

Preferably, tetrabutylammonium bromide is used as a phase transfer catalyst for the Wittig reaction.

Preferably, the solvent for the Wittig reaction comprises at least one selected from the group consisting of N,N-dimethylformamide, dimethyl sulfoxide, and N,N-dimethylacetamide, more preferably N,N-dimethylformamide.

Preferably, the molar ratio of the compound M2, the base, and the phase transfer catalyst is 1:(3 to 5):(0.04 to 0.06).

Preferably, the molar ratio of the compound M2 to ethyltriphenylphosphonium bromide is 1:(1.3 to 1.8), more preferably 1:1.5.

Preferably, the Wittig reaction is carried out by heating to 90 to 110° C. for 15 to 25 h.

Preferably, the compound of Formula I is obtained after the Wittig reaction without further purification.

The present disclosure further provides a method for preparing a compound of Formula II,

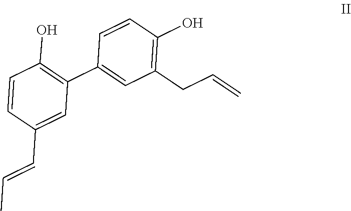 [SMILES: C=CCc1cc(-c2cc(/C=C/C)ccc2O)ccc1O]

comprising the following steps:
(a) subjecting the compound of Formula I prepared by the method mentioned above to an allylation reaction to prepare a compound M3;

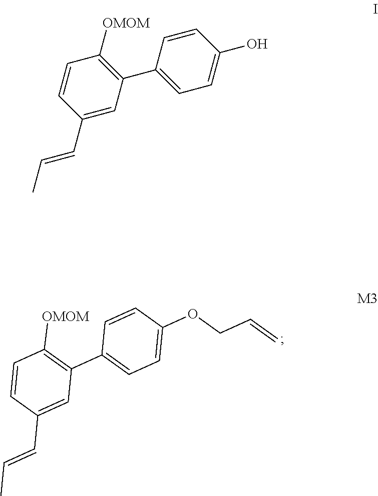

(b) subjecting the compound M3 to MOM deprotection under acidic conditions to prepare a compound M4;

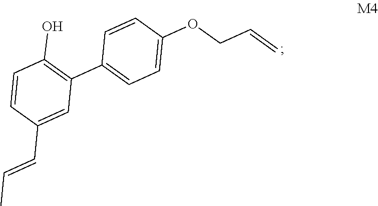 [SMILES: C=CCOc1ccc(-c2cc(/C=C/C)ccc2O)cc1]

(c) subjecting the compound M4 to a rearrangement reaction to prepare the compound of Formula II, i.e., the honokiol derivative.

Preferably, in the allylation reaction of step (a), allyl bromide is used as a reagent; potassium carbonate and/or cesium carbonate is used as a base, and potassium carbonate is more preferable.

Preferably, the molar ratio of the compound of Formula I, the allyl bromide, and the base is 1:(1 to 1.1):(1.5 to 2.2).

Preferably, in step (a), the solvent for the allylation reaction comprises at least one selected from the group consisting of acetone, acetonitrile, and tetrahydrofuran, more preferably acetone.

Preferably, in the allylation reaction of step (a), a reaction temperature is 35 to 50° C., and the reaction time is 5 to 8 h.

Preferably, in step (b), an acid comprises at least one selected from the group consisting of trifluoroacetic acid, p-toluenesulfonic acid, and formic acid, more preferably formic acid.

Preferably, in step (b), the molar ratio of the compound M3 to the acid is 1:(8 to 10).

Preferably, in step (b), the MOM deprotection is carried out at a temperature of 0 to 40° C., more preferably 35 to 40° C., for 15 to 30 h, more preferably 24 h.

Preferably, in step (b), the solvent for the MOM deprotection is dichloromethane.

Preferably, in the rearrangement reaction of step (c), diethylaluminum chloride and/or tin tetrachloride is used as a reagent, and diethylaluminum chloride is more preferable.

Preferably, the molar ratio of the compound M4 to the diethylaluminum chloride is 1:(0.4 to 1.1).

Preferably, in step (c), a solvent for the rearrangement reaction comprises at least one selected from the group consisting of dichloromethane, n-hexane, and toluene, more preferably dichloromethane.

Preferably, in step (c), a reaction temperature is −5° C. to 5° C., and the reaction time is 4 to 10 h.

Preferably, step (c) employs purification methods of trituration and crystallization, more preferably trituration with petroleum ether and crystallization from n-heptane.

The compound of Formula I prepared according to the present disclosure has a trans, E-configuration structure, and the compound of Formula II also has a trans, E-configuration structure.

The present disclosure provides the following advantageous effects.

The starting materials of the methods are inexpensive and readily available, with low cost, simple steps, mild reaction conditions, low purification requirements, high purity, high yield, environmental friendliness, safety, and suitability for industrial production. Therefore the methods have significant application value.

The total yield for preparing the compound of Formula I in the present disclosure is above 55%, with a purity of above 94% (determined by HPLC) without requiring purification.

The total yield for preparing the honokiol derivative in the present disclosure is above 31.5%, with a purity of above 96.5% (determined by HPLC).

### BRIEF DESCRIPTION OF THE DRAWINGS

FIG. 1 shows the ¹H NMR spectrum of compound M2 prepared in Example 1.

FIG. 2 shows the ¹H NMR spectrum of the compound of Formula I prepared in Example 1.

FIG. 3 shows the ¹H NMR spectrum of compound M3 prepared in Example 1.

FIG. 4 shows the ¹H NMR spectrum of the compound of Formula II prepared in Example 1.

FIG. 5 shows the HPLC chromatogram of the compound of Formula I prepared in Example 1.

FIG. 6 shows the HPLC chromatogram of the compound of Formula II prepared in Example 1.

FIG. 7 shows the HPLC chromatogram of the compound of Formula II prepared in Example 2.

### DETAILED DESCRIPTION OF ILLUSTRATED EMBODIMENTS

The present disclosure is described in further detail below by way of examples. The following examples are intended to illustrate the present disclosure exemplarily and are not intended to limit the scope of the present disclosure.

In the examples of the present disclosure, unless otherwise specified, all raw material components are commercially available products well known to those skilled in the art. In the examples of the present disclosure, unless specifically indicated, the technical means used are conventional means well known to those skilled in the art.

The technical concept of the present disclosure lies in using 3-bromo-4-hydroxybenzaldehyde as a starting material, protecting the phenolic hydroxyl group, coupling to form a biphenyl structure, forming an olefin bond via a Wittig reaction, followed by allylation, deprotection, and rearrangement to prepare the honokiol derivative. The reaction scheme is as follows:

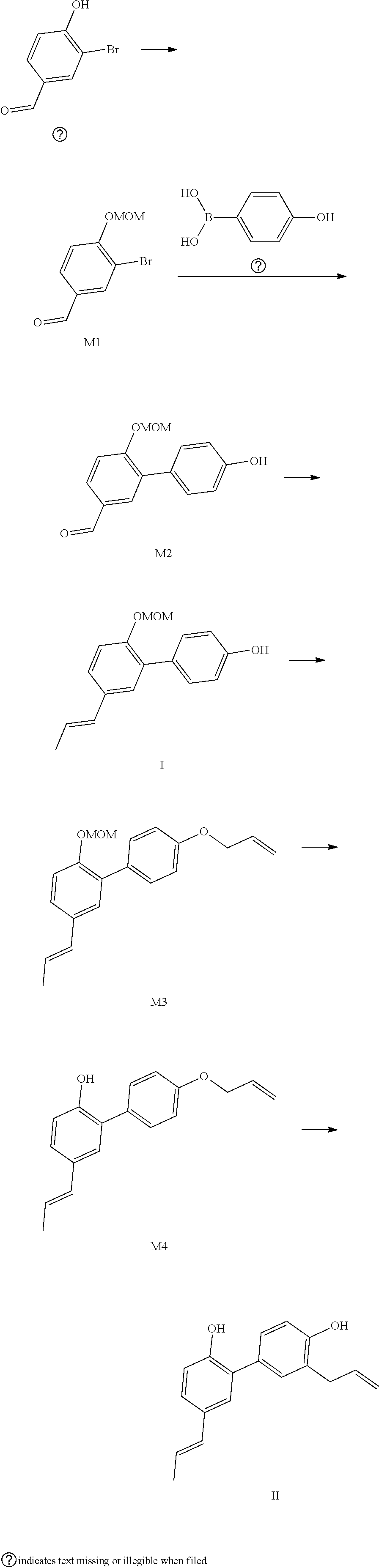

### Example 1

### (1) Synthesis of Compound M1 (3-bromo-4-(methoxymethoxy)benzaldehyde)

3-Bromo-4-hydroxybenzaldehyde (15.00 g, 74.6 mmol), triethylamine (11.30 g, 111.9 mmol), and tetrahydrofuran (90 mL) were added to a reaction flask. The mixture was stirred and cooled to 0° C. Bromomethyl methyl ether (10.25 g, 82.06 mmol) was added dropwise. After the addition was complete, the mixture was stirred at 0° C. for about 4 h. The reaction was stopped upon confirmation by TLC that the 3-bromo-4-hydroxybenzaldehyde was consumed. Water (180 mL) was added to quench the reaction. The mixture was extracted with ethyl acetate (2×100 mL). The combined organic phases were concentrated to a thick paste, which was allowed to stand at room temperature overnight to solidify. The solid was triturated with a mixed solvent of petroleum ether:ethyl acetate=6:1 (v/v) (50 mL), and then was collected by filtration and dried to yield Compound M1 (16.8 g) as a white solid. Yield: 91.9%.

### (2) Synthesis of Compound M2 (4′-hydroxy-6-(methoxymethoxy)-[1,1′-biphenyl]-3-carbaldehyde)

Compound M1 (15.00 g, 61.21 mmol) was added to a reaction flask, followed by tetrahydrofuran (130 mL), water (13 mL), (4-hydroxyphenyl)boronic acid (11.8 g, 85.69 mmol), and potassium carbonate (21.2 g, 153.03 mmol). Stirring was initiated, and palladium(II) acetate (1.4 g, 6.12 mmol) and 1,1′-bis(diphenylphosphino)ferrocene (3.4 g, 6.12 mmol) were added. The flask was evacuated and backfilled with nitrogen three times. The mixture was then heated to 60-65° C. and stirred for 20 h. After confirmation by TLC that Compound M1 was consumed, the mixture was filtered through a pad of diatomaceous earth. Water (390 mL) was added to the filtrate under stirring. The mixture was extracted with dichloromethane (2×200 mL). The combined organic phases were concentrated to dryness. The residue was triturated with a mixed solvent of petroleum ether:ethyl acetate=4:1 (v/v) (30 mL) at room temperature for 2 h to yield Compound M2 (13.2 g) as a light yellow solid. Yield: 83.5%.

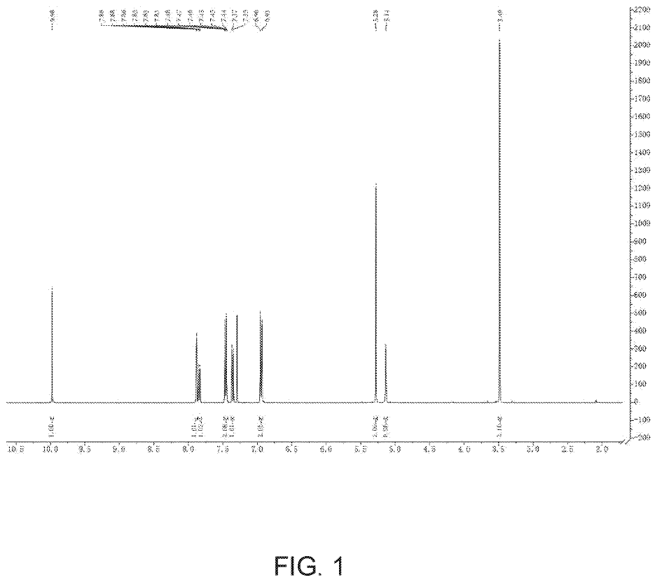

The ¹H NMR spectrum of Compound M2 is shown in FIG. 1. ¹H NMR (400 MHz, CDCl₃): 3.49 (3H, s), 5.14 (1H, s), 5.28 (2H, s), 6.93-6.96 (2H, d), 7.35-7.37 (1H, d), 7.44-7.48 (2H, m), 7.83-7.86 (1H, dd), 7.88 (1H, d), 9.98 (1H, s).

### (3) Synthesis of Compound of Formula I ((E)-2′-(methoxymethoxy)-5′-(prop-1-en-1-yl)-[1,1′-biphenyl]-4-ol)

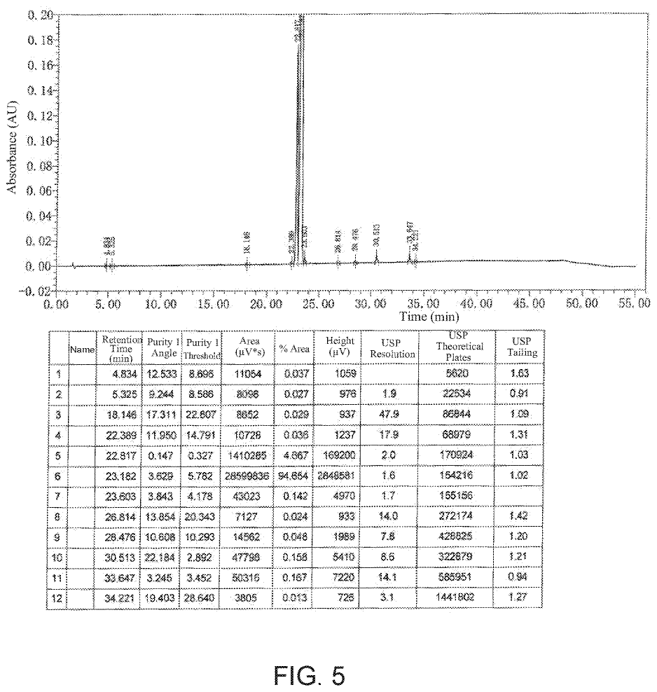

Ethyltriphenylphosphonium bromide (25.87 g, 69.69 mmol), N,N-dimethylformamide (100 mL), and tetrabutylammonium bromide (0.75 g, 2.32 mmol) were added to a reaction flask. The mixture was cooled to 0° C. and stirred for 10 min. Potassium tert-butoxide (20.85 g, 185.84 mmol) was then added, and stirring was continued at 0° C. for 10 min. Finally, Compound M2 (12.00 g, 46.46 mmol) was added. The flask was evacuated and backfilled with nitrogen three times. Under a nitrogen atmosphere, the mixture was allowed to warm to room temperature, stirred for 30 min, and then heated to 95-100° C. for 20 h. After confirmation by TLC that Compound M2 was consumed, the mixture was cooled and slowly poured into water (400 mL). The mixture was extracted with dichloromethane (2×120 mL). The combined organic phases were treated with 1M sodium hydroxide solution (50 mL) and stirred at room temperature for 30 min. Liquid separation was performed. The aqueous layer was adjusted to pH 5-6 with 1M hydrochloric acid and extracted with dichloromethane (2×100 mL). The combined organic phases were concentrated to yield the compound of Formula I (9.2 g) as an oil. Yield: 73.2%. The HPLC chromatogram of the compound of Formula I is shown in FIG. 5, with a purity of 94.65%.

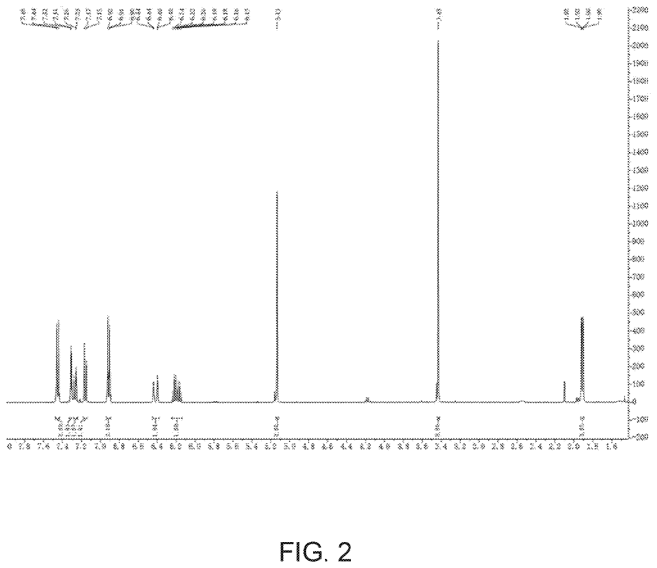

The ¹H NMR spectrum of the compound of Formula I is shown in FIG. 2. ¹H NMR (400 MHz, CDCl₃): 1.90-1.92 (3H, dd), 3.43 (3H, s), 5.13 (2H, s), 6.15-6.24 (1H, m), 6.40-6.44 (1H, dd, J=15.8 Hz), 6.90-6.92 (2H, dd), 7.15-7.17 (1H, d), 7.25-7.28 (1H, dd), 7.31-7.32 (1H, d), 7.44-7.46 (2H, d).

### (4) Synthesis of Compound M3 ((E)-4′-(allyloxy)-2-(methoxymethoxy)-5-(prop-1-en-1-yl)-1,1′-biphenyl)

The compound of Formula I (9.00 g, 33.29 mmol) was added to a flask and dissolved in acetone (40 mL) with stirring. Potassium carbonate (9.2 g, 66.58 mmol) was added, followed by the slow addition of allyl bromide (4.23 g, 34.95 mmol) at room temperature. After the addition was complete, the mixture was heated to 40-45° C. and reacted for about 6 h. After confirmation by TLC that the compound of Formula I was consumed, water (100 mL) was added to quench the reaction. The mixture was extracted with dichloromethane (2×50 mL). The combined organic phases were washed with 10% brine, separated, and concentrated to dryness to yield Compound M3 (9.6 g) as an oil. Yield: 92.8%.

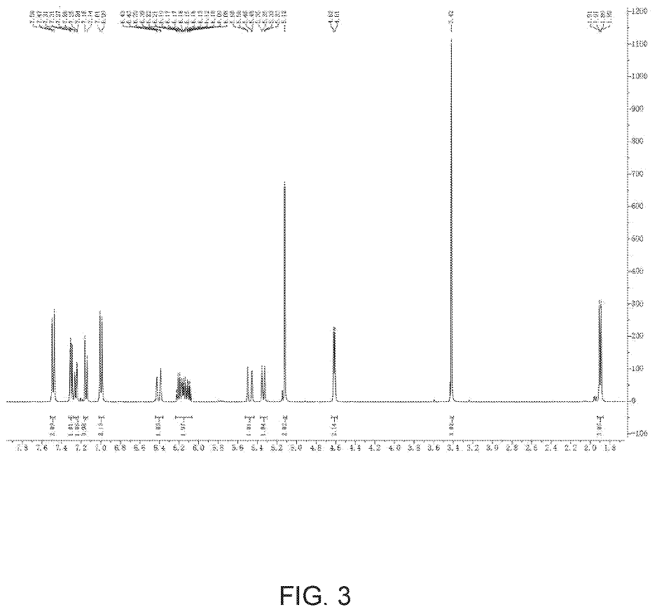

The ¹H NMR spectrum of Compound M3 is shown in FIG. 3. ¹H NMR (400 MHz, CDCl₃): 1.89-1.91 (3H, dd), 3.42 (3H, s), 4.61-4.62 (2H, d), 5.12 (2H, s), 5.33-5.35 (1H, dd), 5.45-5.50 (1H, dd), 6.08-6.22 (2H, m), 6.39-6.43 (1H, dd, J=15.6 Hz), 6.99-7.01 (2H, d), 7.14-7.16 (1H, d), 7.24-7.27 (1H, dd), 7.31 (1H, d), 7.47-7.50 (2H, d).

### (5) Synthesis of Compound M4 ((E)-4′-(allyloxy)-5-(prop-1-en-1-yl)-[1,1′-biphenyl]-2-ol)

Compound M3 (9.00 g, 28.99 mmol) was added to a flask and dissolved in dichloromethane (30 mL). Formic acid (10 mL) was added at room temperature, and the mixture was heated to 35-40° C. and stirred for 24 h. After confirmation by TLC that Compound M3 was consumed, water (15 mL) was added to quench the reaction. Liquid separation was performed. The organic phase was washed with water (2×15 mL), then with saturated sodium bicarbonate solution (10 mL). The organic phase was concentrated to yield Compound M4 (6.1 g) as an oil. Yield: 79%.

### (6) Synthesis of Compound of Formula II ((E)-3′-allyl-5-(prop-1-en-1-yl)-[1,1′-biphenyl]-2,4′-diol)

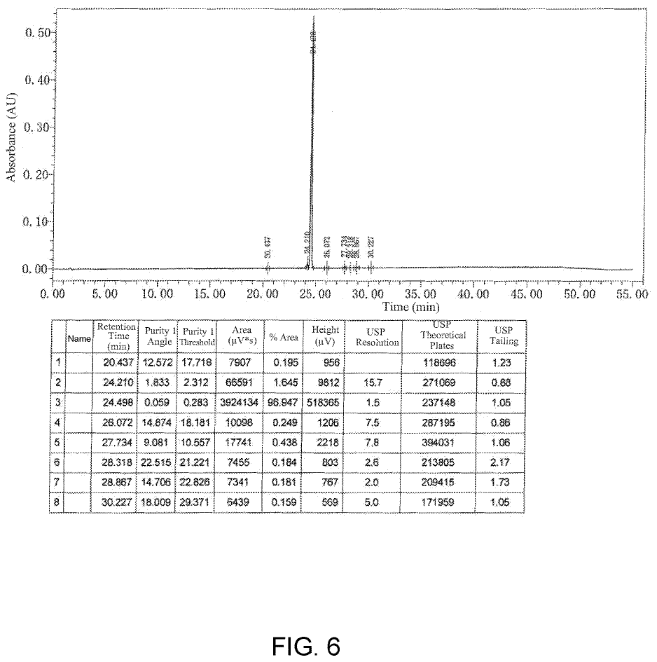

Compound M4 (6.00 g, 22.55 mmol) was dissolved in dichloromethane (60 mL) and cooled to 0° C. Diethylaluminum chloride (1.0 M in hexane, 20 mL) was slowly added. After the addition was complete, the mixture was stirred at 0° C. for about 6 h. After confirmation by TLC that Compound M4 was consumed, water (20 mL) was added to quench the reaction, and then separated layers were obtained. The aqueous layer was extracted with dichloromethane (2×20 mL). The combined organic phases were treated with 1M sodium hydroxide solution (30 mL) and stirred for 30 min, and then separated layers were obtained. The aqueous layer was adjusted to pH 5 with 1M hydrochloric acid and extracted with dichloromethane (2×50 mL). The combined organic phases were dried, concentrated to dryness, and triturated with petroleum ether at room temperature for 2 h to obtain a solid. The solid was collected by filtration. The filter cake was dissolved in n-heptane (85 mL) by heating to reflux, and then cooled to precipitate the solid. The solid was collected by filtration and dried to yield the compound of Formula II (4.6 g). Yield: 76.6%. The HPLC chromatogram of the compound of Formula II is shown in FIG. 6, with a purity of 96.95%.

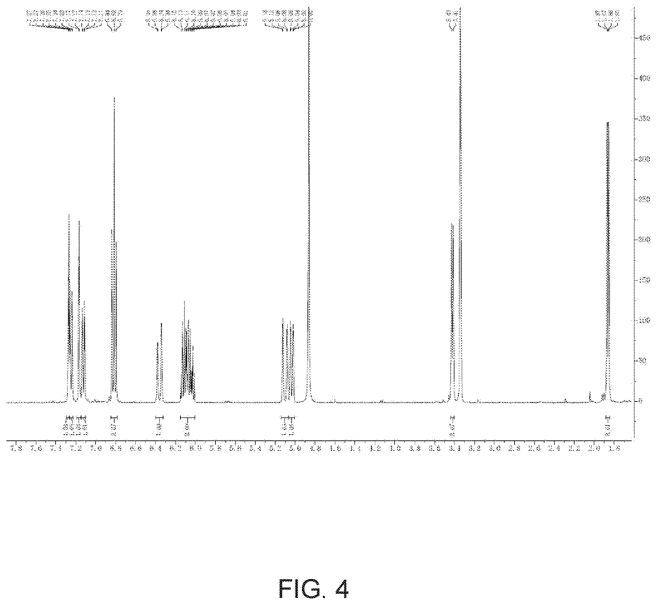

The ¹H NMR spectrum of the compound of Formula II is shown in FIG. 4. ¹H NMR (400 MHz, MeOD): 1.85-1.87 (3H, dd), 3.41-3.43 (2H, d), 5.02-5.05 (1H, dd), 5.08-5.13 (1H, dd), 6.01-6.15 (2H, m), 6.34-6.38 (1H, dd, J=15.8 Hz), 6.79-6.84 (2H, t), 7.11-7.14 (11H, dd), 7.17 (11H, d), 7.23-7.26 (1H, dd), 7.27 (1H, d).

### Example 2

### (1) Synthesis of Compound M1 (3-bromo-4-(methoxymethoxy)benzaldehyde)

3-Bromo-4-hydroxybenzaldehyde (200.00 g, 0.995 mol), triethylamine (150.9 g, 1.492 mol), and tetrahydrofuran (800 mL) were added to a reaction flask. The mixture was stirred and cooled to 0° C. Bromomethyl methyl ether (136.7 g, 1.094 mol) was added dropwise. After the addition was complete, the mixture was stirred at 0° C. for about 5 h. The reaction was stopped upon confirmation by TLC that the 3-bromo-4-hydroxybenzaldehyde was consumed. Water (1.6 L) was added to quench the reaction. The mixture was extracted with ethyl acetate (2×1 L). The combined organic phases were concentrated to a thick paste. The paste was allowed to stand at room temperature overnight to solidify. The solid was triturated with a mixed solvent of petroleum ether:ethyl acetate=6:1 (v/v) (400 mL), and then was collected by filtration and dried to yield Compound M1 (226.3 g) as a white solid. Yield: 92.8%.

### (2) Synthesis of Compound M2 (4′-hydroxy-6-(methoxymethoxy)-[1,1′-biphenyl]-3-carbaldehyde)

Compound M1 (220.00 g, 0.898 mol) was added to a reaction flask, followed by tetrahydrofuran (1.3 L), water (130 mL), (4-hydroxyphenyl)boronic acid (173.4 g, 1.257 mol), and potassium carbonate (310.3 g, 2.245 mol). Stirring was initiated, and palladium(II) acetate (20.2 g, 0.089 mol) and 1,1′-bis(diphenylphosphino)ferrocene (49.3 g, 0.089 mol) were added. The flask was evacuated and backfilled with nitrogen three times. The mixture was then heated to 60-65° C. and stirred for 22 h. After confirmation by TLC that Compound M1 was consumed, the mixture was filtered through a pad of diatomaceous earth. Water (2.6 L) was added to the filtrate under stirring. The mixture was extracted with dichloromethane (2×1 L). The combined organic phases were concentrated to dryness. The residue was triturated with a mixed solvent of petroleum ether:ethyl acetate=4:1 (v/v) (600 mL) at room temperature for 2 h to yield Compound M2 (188.7 g) as a light yellow solid. Yield: 81.4%.

### (3) Synthesis of Compound of Formula I ((E)-2′-(methoxymethoxy)-5′-(prop-1-en-1-yl)-[1,1′-biphenyl]-4-ol)

Ethyltriphenylphosphonium bromide (366.4 g, 0.987 mol), N,N-dimethylformamide (1.5 L), and tetrabutylammonium bromide (10.6 g, 32.9 mmol) were added to a reaction flask. The mixture was cooled to 0° C. and stirred for 10 min. Potassium tert-butoxide (295.3 g, 2.632 mol) was then added, and stirring was continued at 0° C. for 10 min. Finally, Compound M2 (170.00 g, 0.658 mol) was added. The flask was evacuated and backfilled with nitrogen three times. Under a nitrogen atmosphere, the mixture was allowed to warm to room temperature, stirred for 30 min, and then heated to 95-100° C. for 22 h. After confirmation by TLC that Compound M2 was consumed, the mixture was cooled and slowly poured into water (5 L). The mixture was extracted with dichloromethane (3×1 L). The combined organic phases were treated with 1M sodium hydroxide solution (700 mL) and stirred at room temperature for 30 min. Liquid separation was performed. The aqueous layer was adjusted to pH 5-6 with 1M hydrochloric acid and extracted with dichloromethane (2×1 L). The combined organic phases were concentrated to yield the compound of Formula I (131.4 g) as an oil. Yield: 73.8%.

### (4) Synthesis of Compound M3 ((E)-4′-(allyloxy)-2-(methoxymethoxy)-5-(prop-1-en-1-yl)-1,1′-biphenyl)

The compound of Formula I (130.00 g, 0.481 mol) was added to a flask and dissolved in acetone (600 mL) with stirring. Potassium carbonate (132.9 g, 0.962 mol) was added, followed by the slow addition of allyl bromide (61.1 g, 0.505 mol) at room temperature. After the addition was complete, the mixture was heated to 40-45° C. and reacted for about 7 h. After confirmation by TLC that the compound of Formula I was consumed, water (1.5 L) was added to quench the reaction. The mixture was extracted with dichloromethane (2×800 mL). The combined organic phases were washed with 10% brine, separated, and concentrated to dryness to yield Compound M3 (143.6 g) as an oil. Yield: 96.2%.

### (5) Synthesis of Compound M4((E)-4′-(allyloxy)-5-(prop-1-en-1-yl)-[1,1′-biphenyl]-2-ol)

Compound M3 (140.00 g, 0.451 mol) was added to a flask and dissolved in dichloromethane (500 mL). Formic acid (170 mL) was added at room temperature, and the mixture was heated to 35-40° C. and stirred for 24 h. After confirmation by TLC that Compound M3 was consumed, water (250 mL) was added to quench the reaction. Liquid separation was performed. The organic phase was washed with water (2×250 mL), then once with saturated sodium bicarbonate solution (200 mL). The organic phase was concentrated to yield Compound M4 (97.5 g) as an oil. Yield: 81.2%.

### (6) Synthesis of Compound of Formula II ((E)-3′-allyl-5-(prop-1-en-1-yl)-[1,1′-biphenyl]-2,4′-diol)

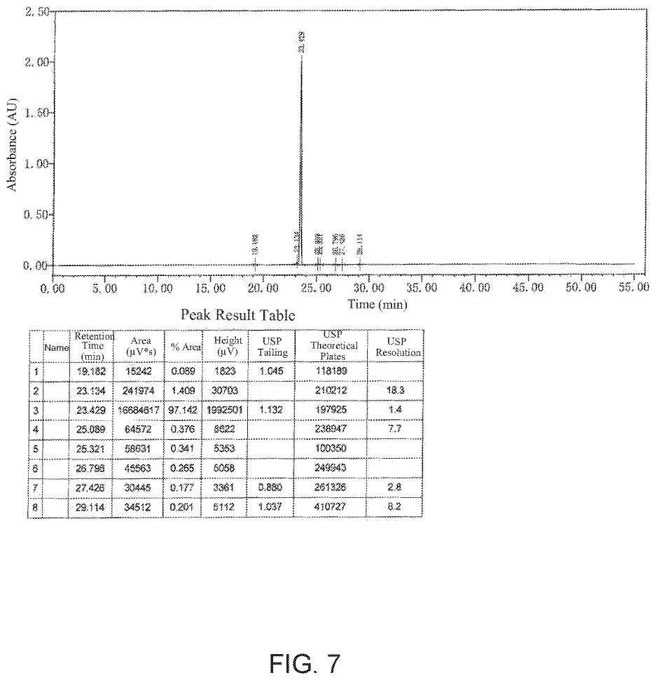

Compound M4 (95.00 g, 0.357 mol) was dissolved in dichloromethane (500 mL) and cooled to 0° C. Diethylaluminum chloride (1.0 M in hexane, 150 mL) was slowly added. After the addition was complete, the mixture was stirred at 0° C. for about 8 h. After confirmation by TLC that Compound M4 was consumed, water (200 mL) was added to quench the reaction, and then separated layers were obtained. The aqueous layer was extracted with dichloromethane (2×500 mL). The combined organic phases were treated with 1M sodium hydroxide solution (1 L) and stirred for 30 min, and then separated layers were obtained. The aqueous layer was adjusted to pH 5 with 1M hydrochloric acid and extracted with dichloromethane (2×500 mL). The combined organic phases were dried, concentrated to dryness, and triturated with petroleum ether at room temperature for 2 h to obtain a solid. The solid was collected by filtration. The filter cake was dissolved in n-heptane (1.2 L) by heating to reflux, and then cooled to precipitate the solid. The solid was collected by filtration and dried to yield the compound of Formula II (75.4 g). Yield: 79.4%. The HPLC chromatogram of the compound of Formula II is shown in FIG. 7, with a purity of 97.14%.

It is necessary to point out that the above examples are only intended to further illustrate and explain the technical solution of the present disclosure, and are not intended to further limit the technical solution of the present disclosure. The preferred embodiments are not intended to limit the scope of protection of the present disclosure. Any modifications, equivalent substitutions, improvements, etc. made within the spirit and principle of the present disclosure shall be included within the scope of protection of the present disclosure.

What is claimed is:

1. A method for preparing a compound of Formula I, 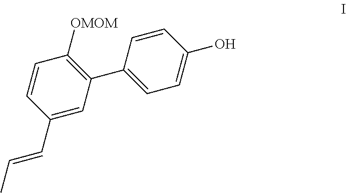
comprising: subjecting a compound M2 as below to a Wittig reaction with ethyltriphenylphosphonium bromide under basic conditions to prepare the compound of Formula I;

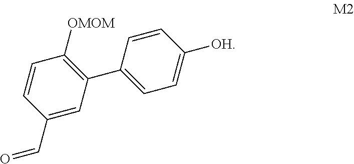

2. The method according to claim 1, wherein the compound M2 is prepared by the following steps:
(1) reacting a compound SM1 with bromomethyl methyl ether to prepare a compound M1; 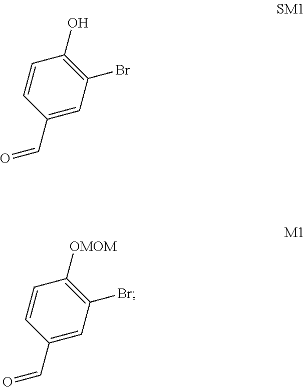
(2) reacting the compound M1 with a compound SM2 in a coupling reaction to prepare the compound M2;

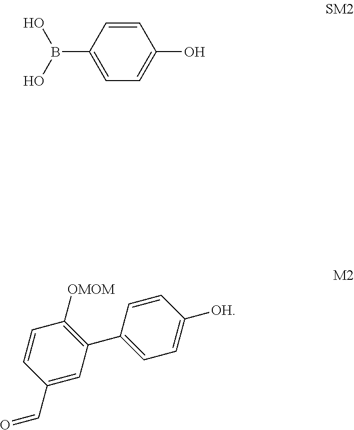

3. The method according to claim 2, wherein in a reaction of step (1), triethylamine is used as a base, and tetrahydrofuran is used as a solvent; a molar ratio of the compound SM1, the bromomethyl methyl ether, and the base is 1:1.0 to 1.4:1.5 to 2.0; a reaction temperature is −5° C. to 5° C., and a reaction time is 3 to 6 h.

4. The method according to claim 2, wherein in the coupling reaction of step (2), Pd(OAc)₂ is used as a catalyst, 1,1′-bis(diphenylphosphino)ferrocene is used as a ligand, and a base comprises at least one selected from the group consisting of potassium carbonate, cesium carbonate and potassium acetate; a molar ratio of the compound M1, the base, the catalyst, and the ligand is 1:2 to 3:0.09 to 0.12:0.09 to 0.12; and a molar ratio of the compound M1 to the compound SM2 is 1:1.0 to 1.5.

5. The method according to claim 2, wherein in the coupling reaction of step (2), a solvent comprises a mixed solvent of N,N-dimethylformamide and water, a mixed solvent of 1,4-dioxane and water, or a mixed solvent of tetrahydrofuran and water; a coupling reaction temperature is 50 to 70° C., and a coupling reaction time is 15 to 25 h.

6. The method according to claim 1, wherein the base comprises at least one selected from the group consisting of potassium tert-butoxide, sodium tert-butoxide, and lithium diisopropylamide; tetrabutylammonium bromide is used as a phase transfer catalyst for the Wittig reaction, and a solvent in the Wittig reaction comprises at least one selected from the group consisting of N,N-dimethylformamide, dimethyl sulfoxide, and N,N-dimethylacetamide; a molar ratio of compound M2, the base, and the phase transfer catalyst is 1:3 to 5:0.04 to 0.06; a molar ratio of the compound M2 to the ethyltriphenylphosphonium bromide is 1:1.3 to 1.8; and the Wittig reaction is carried out by heating to 90 to 110° C. for 15 to 25 h.

7. A method for preparing a compound of Formula II, 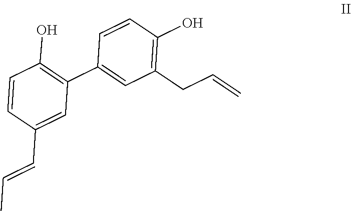 [SMILES: C=CCc1cc(-c2cc(/C=C/C)ccc2O)ccc1O]
comprising the following steps:
(a) subjecting the compound of Formula I prepared by the method according to claim 1 to an allylation reaction to prepare a compound M3; 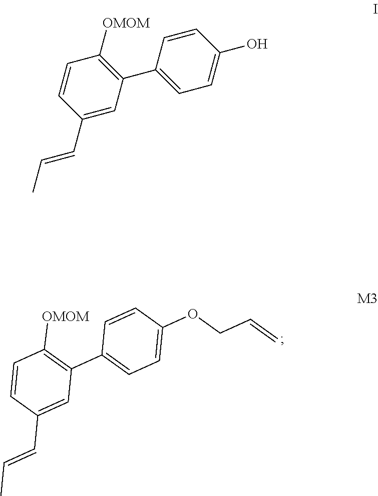
(b) subjecting the compound M3 to MOM deprotection under acidic conditions to prepare a compound M4; 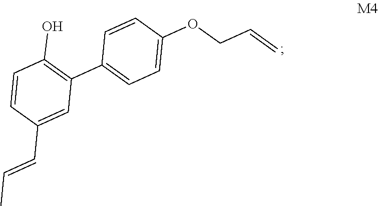 [SMILES: C=CCOc1ccc(-c2cc(/C=C/C)ccc2O)cc1]
(c) subjecting the compound M4 to a rearrangement reaction to prepare the compound of Formula II.

8. The method according to claim 7, wherein in the allylation reaction of step (a), allyl bromide is used as a reagent, potassium carbonate and/or cesium carbonate is used as a base, and a solvent comprises at least one selected from the group consisting of acetone, acetonitrile, and tetrahydrofuran; a molar ratio of the compound of Formula I, the allyl bromide, and the base is 1:1 to 1.1:1.5 to 2.2; a reaction temperature is 35 to 50° C., and a reaction time is 5 to 8 h.

9. The method according to claim 7, wherein in the MOM deprotection of step (b), an acid comprises at least one selected from the group consisting of trifluoroacetic acid, p-toluenesulfonic acid, and formic acid; a molar ratio of the compound M3 to the acid is 1:8 to 10; the MOM deprotection is carried out at a temperature of 0 to 40° C. for 15 to 30 h, with a solvent of dichloromethane.

10. The method according to claim 7, wherein in the rearrangement reaction of step (c), diethylaluminum chloride and/or tin tetrachloride is used as a reagent, and a solvent comprises at least one selected from the group consisting of dichloromethane, n-hexane, and toluene; a molar ratio of the compound M4 to the diethylaluminum chloride is 1:0.4 to 1.1; a reaction temperature is −5° C. to 5° C., and a reaction time is 4 to 10 h.
